(19) United States
(12) Patent Application Publication
(10) Pub. No.: US 2017/0303911 A1
(43) Pub. Date: Oct. 26, 2017

(54) ARTICLE OF MANUFACTURE AND METHOD FOR INCURSION CLOSURE

(71) Applicant: Lee L. Thibodeau, Cumberland, ME (US)

(72) Inventor: Lee L. Thibodeau, Cumberland, ME (US)

(21) Appl. No.: 15/498,453

(22) Filed: Apr. 26, 2017

Related U.S. Application Data
(60) Provisional application No. 62/327,982, filed on Apr. 26, 2016.
(60) Provisional application No. 62/357,364, filed on Jun. 30, 2016.

(51) Int. Cl. A61B 17/04; A61B 17/00; A61B 17/06

(52) U.S. Cl. CPC A61B 17/0401; A61B 17/06166; A61B 17/0487; A61B 17/00491; A61B 2017/0406; A61B 2017/0464

(57) ABSTRACT

An article of manufacture in the form of an incursion repair material arranged to establish a watertight seal about an incursion such as a durotomy and secured in place by a securing mechanism. The incursion material may be graft material and/or a flange of selectable shape and size and made of a material suitable to remain in position on the incursion. The incursion repair material is used in a surgical procedure to seal the incursion. The material may be secured in place with one or more sutures only. It may also be secured in place with devices anchored in underlying substrate, such as bone or scar tissue.

### CROSS REFERENCE TO RELATED APPLICATION

This application is a nonprovisional and claims the priority benefit of U.S. provisional patent application Ser. No. 62/327,982 filed Apr. 26, 2016, entitled Article of Manufacture and Method for Incursion Closure. This application also is a nonprovisional and claims the priority benefit of U.S. provisional patent application Ser. No. 62/357,364 filed Jun. 30, 2016, entitled Article of Manufacture and Method for Incursion Closure. The content of those applications is incorporated herein by reference.

### BACKGROUND OF THE INVENTION

### 1. Field of the Invention

The present invention relates to materials and methods used to close incursions such as durotomies. More particular, the present invention relates to articles of manufacture and methods used to support sutured surfaces of the matter surrounding incursions such as the dura.

### 2. Background of the Invention

The dura is the outermost and most fibrous of the three membranes that cover the brain and the spinal cord. In the course of carrying out brain or spinal surgery, incursion of the dura may be required to access matter covered by the dura. It is also possible that the dural sac may be torn in the course of surgery. The dural incision or the dural tear must be repaired.

A durotomy is a hole in the dura, intended or not, that can be opened during surgical procedures such as spine surgical procedures. Frequently, the dura is very friable and difficult to repair with standard suture techniques. If the dura is not closed in a watertight fashion it can lead to significant postoperative complications which translates into increased patient disability and costs to the system. What is needed is an article of manufacture and a method to aid in the repair of durotomies.

### SUMMARY OF THE INVENTION

The present invention is an article of manufacture and a related method for repairing durotomies. While the focus of the description of the present invention is that of an article of manufacture employed to aid in the repair of durotomies, it is not limited to that application and may be used to enable closure of incursions not limited to those of the dura. The invention is a dural closure that is a flange, which may also be referred to as a button, arranged in combination with suture material to solve the problem of difficult incursion closure such as durotomy closure. The flange may or may not be joined to an underlying substrate using a joining device that is located at least in part under the dura. The flange functions as a layer to buttress/support the surface of the matter surrounding the wound, such as the dura in relation to a durotomy. The flange allows a surgeon to achieve a watertight dural closure without the usual suture techniques. It also allows the surgeon to secure the suture in a much easier and user friendly manner. The several methods for securing the flange include those for which no suture knotting is required. This is especially important as surgical procedures become more minimally invasive. Furthermore, the flange allows the surgeon to apply a glue, sealant, patch and/or graft to the dural surface and approximate it and hold it securely with structural support from the flange to maintain it in position.

The invention is further described with respect to the following detailed description, accompanying drawings and the appended claims.

### BRIEF DESCRIPTION OF THE DRAWINGS

FIG. 1 is a side view of a first embodiment of the flange of the present invention.

FIG. 2 is a side view of a second embodiment of the flange of the present invention.

FIG. 3A is a side view of the flange of the present invention joined to the dura with a suture.

FIG. 3B is a top view of the flange of the present invention joined to the dura with a suture.

FIG. 3C is a side view of the flange of the present invention joined to the dura with a bushing wherein the bushing is shown on a suture prior to contact with the flange.

FIG. 3D is a side view of the present invention of FIG. 3C with the bushing in position on the flange.

FIG. 4 is a side view of the flange of the present invention joined to the dura with a first alternative joining device.

FIG. 5 is a side view of the flange of the present invention with a second alternative joining device.

FIG. 6 is a side view of a third embodiment of the flange of the present invention with a third alternative joining device.

FIG. 7 is a side view of the third flange of the present invention with a fourth alternative joining device.

FIG. 8 is a side view of the third flange of the present invention with a fifth alternative joining device.

FIG. 9 is a side view of the third flange of the present invention with a sixth alternative joining device.

FIG. 10 is a side view of a first alternative arrangement for repairing a dural defect.

FIG. 11 is a side view of a second alternative arrangement for repairing a dural defect.

### DETAILED DESCRIPTION OF THE INVENTION

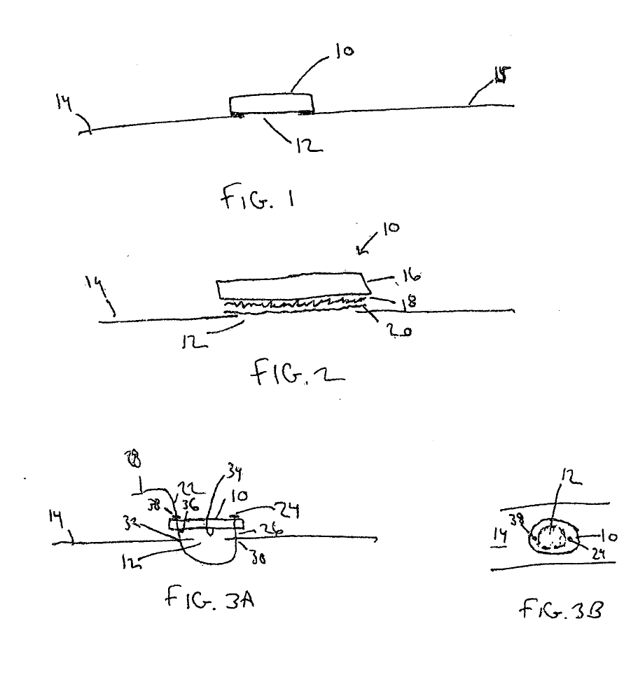

FIGS. 1 and 2 show a first embodiment of a flange 10 of the present invention positioned on a durotomy 12 of a dura 14. It is to be understood that the flange 10 may be used to cover other incursions not limited to the dura 14. The flange 10 may be fabricated of any material suitable for the environment and to seal the durotomy 12. For example, the flange 10 may be formed of an absorbent material, a nonabsorbent material or a combination of the two. It may be flexible or relatively inflexible. It may be made of a nonmetallic material including of a viscoelastic or other material. It may be made of an open-cell foam, a closed-cell foam or a solid material. It may be formed of a single layer of material or a plurality of layers of material. If made of a plurality of layers, each of the layers may be made of the same or different materials. DuraSeal provided by Integra may be used as part of the repair procedure for this version of the present invention as well as the other apparatuses described herein. The flange 10 may be shaped and sized suitable for the intended purposes. For example, the flange 10 may be round, rectangular or customized shape. Its thickness is selectable provided it is not too thick or too thin so as to comprise the desired watertight seal of the durotomy 12.

FIG. 2 shows a second embodiment of the flange 10 as a combination of optional components, wherein the flange is primary component 16 having positioned between it and the dura 14, a first supplemental component 18 and second supplemental component 20. The first supplemental component 18 may be a gel foam or a graft, for example, while the second supplemental component 20 may be a sealant or a glue, for example, to secure the first supplemental component 18 to the dura 14. Optionally, only one of the two supplemental components 18 and 20 may be used in combination with the primary flange component 16. It is also to be noted that the flange 10 itself may be formed of either or both of the first supplemental component 18 and the second supplemental component 20 as a function of the desired characteristics of the flange 10 for the purpose of sealing the durotomy 12 or such other incision as is to be sealed.

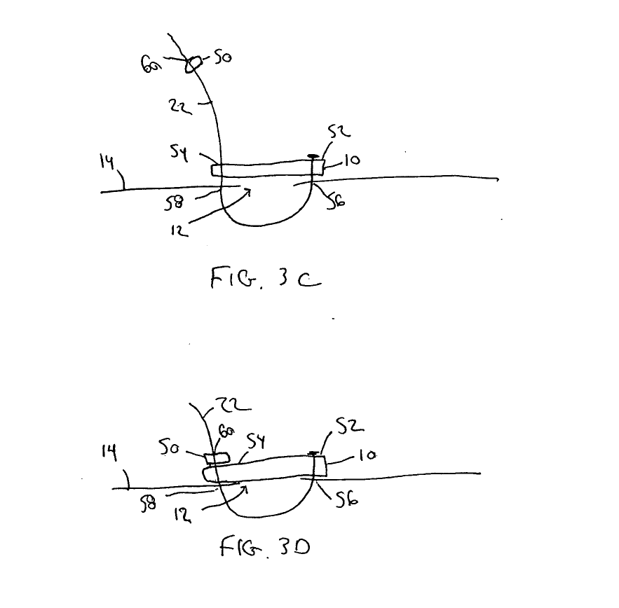

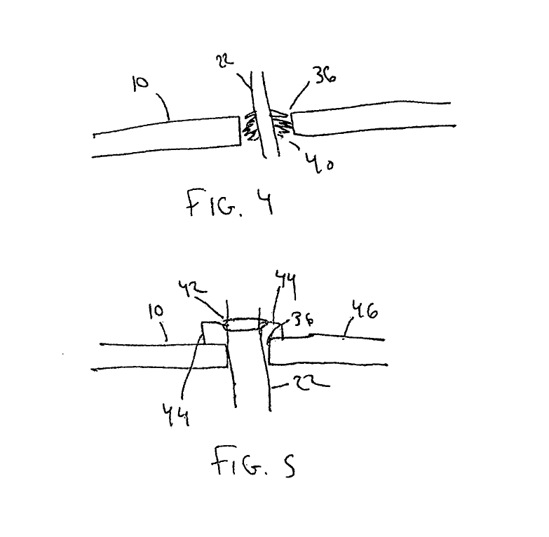

As shown in FIGS. 3A and 3B, the flange 10 of the first embodiment may be applied to the dura 14 over the durotomy 12 using a suture 22. The flange 10 may be secured with an extended tip 24 or a knot of the suture 22 at first end 26. A second end 28 of the suture 22 is inserted into the dura 14 at location 30 at the perimeter of the durotomy 12 directed under the durotomy 12 and directed out of the dura 14 at location 32 through port 36 of the flange 10. The suture 22 at that location of port 36 may be secured to the flange 10 with extended tip 38 or a knot of the suture 22. The flange 10 is thereby drawn onto the surface of the dura 14 over the durotomy 12, with its size sufficient to completely cover the durotomy 12 as shown in FIG. 3B and establish a watertight seal between the flange 10 and dura 14. Underside 34 of the flange 10 may include an adhering material or one or both of supplemental components 18 and 20 previously described to aid in sealing the durotomy 12.

An alternative form of this version of the invention is illustrated in FIGS. 3C and 3D. The flange 10 of the first embodiment may be applied to the dura 14 over the durotomy 12 using a suture 22. The flange 10 may be secured to the dura 14 using a suture 22 and a frictional fitting such as a crimping or bushing 50 that may be used to retain the flange 50 in place on the dura 12 over the durotomy 14 at either or both of first end 52 and second end 54 of the flange 10. Use of the bushing 50 ensures that the flange 10 is secured in place without the need to make a knot that may or may not maintain the flange 50 in place as the wound heals. As shown in FIG. 3C, the flange 10 is secured in place over the durotomy 14 by passing the suture 22 through the first end 52 of the flange 10, down through the dura 12 at site 56, up through the dura 12 and the second end 54 of the flange 10 at site 58 and secured in place on the surface of the flange 50 at the second end 54. The suture 22 may be secured on the flange 50 at the first end 52 with either a knot or a second bushing. The needle is passed through the bushing 50 after exiting the flange 50 at second end 54 and the bushing is pushed down along the suture 22 until it makes contact with the flange 10 at the second end 54, as shown in FIG. 3D. The suture 22 may then be cut off with the bushing 50 acting to secure the end of the suture 22, making it unnecessary for the surgeon to tie a knot. This arrangement does not require making any knots in the suture material to secure the flange 10. The bushing 50 can be sized and shaped to provide a substantial coverage of an area of the flange 10 without compromising the integrity of the flange 10 while also providing an effective retention of the flange 10 on the dura 14. The bushing 50 may be made of a dissolvable material. It may be a nonmetallic material such as a viscoelastic material such as a rubber material having a relatively high coefficient of friction. It may be of selectable size and include a portal 60 sized to allow the needle and the suture 22 to pass through it with effort but that will not otherwise easily slide along on the suture 22. The flange 10 is thereby retained on the surface of the dura 14 over the durotomy 12, with its size sufficient to completely cover the durotomy 12 as shown in FIG. 3D and establish a watertight seal between the flange 10 and dura 14. Underside 34 of the flange 10 may include an adhering material or one or both of supplemental components 18 and 20 previously described to aid in sealing the durotomy 12.

A first alternative flange joining device for securing the flange 10 in place on the dura 14 is a serrated or toothed extension 40 placed on the suture 22 and arranged to reside in port 36 of the flange 10 when the flange 10 has been positioned where desired as shown in FIG. 4. The extension 40 is arranged to prevent movement of the flange 10 in a manner that breaks the watertight seal between the flange 10 and the dura 14. A second alternative flange joining device for securing the flange 10 in place on the dura 14 is shown in FIG. 5. The second alternative joining device is a crimp ring 42 secured by posts 44 established on surface 46 of the flange 10. The crimp ring 42 crimps the suture 22 in place when clamped so as to secure the flange 10 in position on the dura.

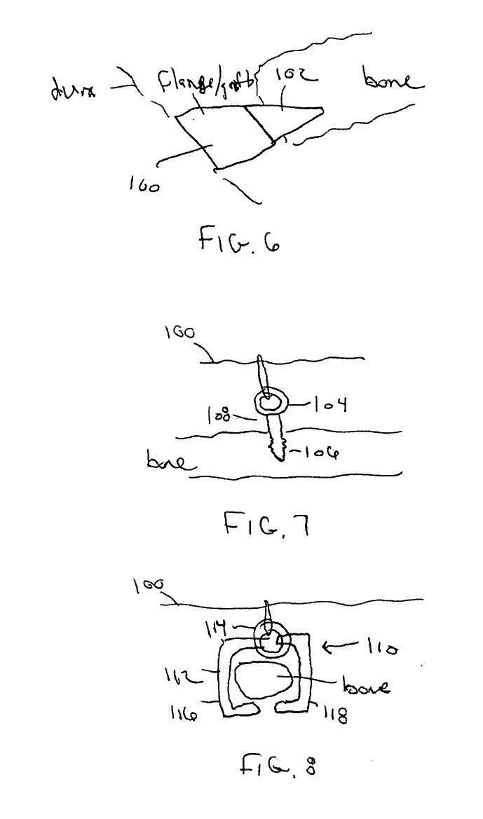

While the flange 10 shown includes the port 36 used to insert the suture 22 at the specific location of the port 36, a third flange embodiment represented as flange 100 shown in FIGS. 6-9 does not include a port to enable the securing of the flange 100 to the dura. The flange is primarily or entirely secured to an underlying substrate, such as bone or a scar, for example, rather than to the dura. The flange 100 may be of selectable shape and size. The mechanism for securing the flange 100 to an underlying substrate does not require the use of a port. As a result, it may be placed at any location of interest.

A third alternative joining device for securing the flange 100 in place on the dura is a pointed insertion member 102 is shown in FIG. 6. The insertion member 102 is coupled to the flange 100, such as with an adhesive, for example. The insertion member 102 is of sufficient length to pierce underlying bone or scar material and remain embedded therein long enough for the flange 100 to establish desired growth of the dura at the incursion. The insertion member 102 may be made of metallic or nonmetallic material that must be ridge enough to pierce the bone and flexible enough not to break off while in service. The flange 100, which may include graft material, in combination with the insertion member 102 provides a mechanism for securing the dural defect without the need to add sutures, although sutures may also be used to further secure the flange 100 in place.

A fourth alternative joining device for securing the flange 100 in place on the dura is a substrate screw 104 shown in FIG. 7. The screw 104 includes a first end 106 and a second end 108. The first end 106 is threaded or otherwise configured to be joined to the underlying substrate, such as a local bone. The second end 108 includes an eyelet or other type of configuration to enable the securing of a suture between the screw 104 and the flange 100. The screw 104 thereby provides an anchor point for the flange 100 that is more difficult to loosen or come apart than the use of a suture alone through the dura. The screw 104 may be screwed directly into the local substrate. The local substrate, particularly if it is bone material, may be drilled to establish a port for insertion of the first end 106 therein.

A fifth alternative joining device for securing the flange in place on the dura is a clip 110 shown in FIG. 8. The clip 110 includes a clip structure 112 and an eyelet 114. The clip structure 112 includes a first leg 116 and a second leg 118. The first leg 116 and the second leg 118 may be joined together by the eyelet 114 therebetween. Alternatively, the clip structure 112 may be a unitary member with the eyelet 114 affixed at the closed end thereof. The open end of the clip structure 112 is of adjustable spacing, such as by the closed end being hinged. The clip 110 may be opened wide enough at the open end to be placed around the underlying substrate, such as a local bone. The applied force may then be released so that the clip 110 is engaged about the underlying substrate. The eyelet 114 or another type of retaining configuration is arranged to enable the securing of a suture between the clip 110 and the flange 100. The clip 110 thereby provides an anchor point for the flange 100 that is more difficult to loosen or come apart than the use of a suture alone through the dura.

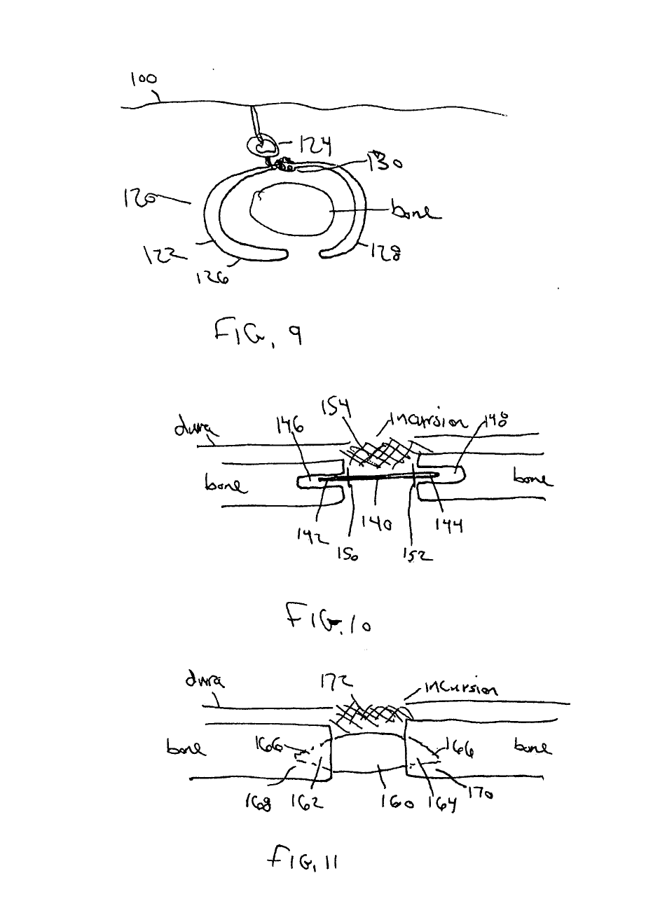

A sixth alternative joining device for securing the flange 100 in place on the dura is a clamp 120 shown in FIG. 9. The clamp 120 includes a clamp structure 122 and an eyelet 124. The clamp structure 122 includes a first leg 126 and a second leg 128. The first leg 126 and the second leg 128 may be joined together by a spring 130 that is loaded to maintain the clamp 120 in a closed position. The eyelet 124 affixed to the clamp structure 122 near the spring 130. The clamp 120 may be opened wide enough at the open end to be placed around the underlying substrate, such as a local bone. The applied force may then be released so that the clamp 120 clamps onto the underlying substrate. The eyelet 124 or another type of retaining configuration is arranged to enable the securing of a suture between the clamp 120 and the flange 100. The clamp 120 thereby provides an anchor point for the flange 100 that is more difficult to loosen or come apart than the use of a suture alone through the dura.

It is noted that other mechanisms may be used to secure either a flange, graft material or both at the dural defect area. For example, one or more staples may be applied to underlying substrate material, such as a scar or a bone. The staples may be configured to allow for insertion of one or more sutures thereunder so that the staples secure the sutures in place, with the sutures securing the flange and/or graft material to the dural defect. In addition, bone awls and/or punches may be used to create portals for optimal placement at locations of interest for bone support devices of the type described herein as well as sutures.

Alternative arrangements for repairing a dural defect is shown in FIGS. 10 and 11. Both apparatuses are inserted into underlying substrate, such as bone, to establish a foundation in the vicinity of a dural defect. The foundation is used to secure flange and/or graft material thereto and thereon, which flange and/or graft material may be built up from the underlying substrate to fill in the defect. The first alternative apparatus of FIG. 10 is a bar or stick 140 having a first end 142 and a second end 144. Each of the first end 142 and the second end 144 is configured for insertion into and retention by substrate material, such as at ports 146 and 148, respectively. The ports 146 and 148 may be established in the substrate such as by drilling, with an awl or with a bone punch. The stick 140 includes retainers 150 and 152 to ensure that the ends 142 and 144 will not slip out of ports 146 and 148. The stick 140 may be spring-loaded to accommodate flexure of the stick 140 and it may include crimps or other surface texture to aid in retaining graft material 154 thereto. Once the stick 140 has been established in the bone, the graft material 154 and/or other dural defect repair materials may be applied to the stick 140 with sufficient thickness to keep the material 154 in place at least until the defect has been repaired.

The second alternative apparatus of FIG. 11 is a board 160 having a first end 162 and a second end 164. Each of the first end 162 and the second end 164 includes an insertion member 166 similar to the point insertion member 102 shown in FIG. 6. The insertion members 166 are configured for insertion into and retention by substrate material, such as at locations 168 and 170, respectively. The insertion members 166 are of sufficient length to pierce underlying bone or scar material and remain embedded therein long enough for the flange board 160 to establish desired growth of the dura at the incursion. The insertion members 166 may be made of metallic or nonmetallic material that must be ridge enough to pierce the bone and flexible enough not to break off while in service. An awl or punch may be used to facilitate insertion of the board insertion members 166 in the underlying substrate. The board 160 may be spring-loaded to accommodate flexure of the board 160 and it may include crimps or other surface texture to aid in retaining graft material 172 thereto. Once the board 160 has been established in the underlying substrate, the graft material 172 and/or other dural defect repair materials may be applied to the board 160 with sufficient thickness to keep the material 172 in place at least until the defect has been repaired.

The components of the apparatuses described herein, as well as tools useful to place them where desired, may be provided in one or more kits suitable for use in an operating environment. The kits may be created based on the reuse or one-time use of the components. They may also be established based on the particular type or types of flange/graft retention mechanism desired. For example, there may be an instrument tray with specifically designed instruments as well as any standard instruments of utility. There may also be a disposable tray with components specifically used for insertion to repair the incursion, such as, for example, one or more of the securing mechanisms described herein.

It is to be understood that other mechanisms may be employed to secure the flange in a manner to maintain its watertight seal of the dura at the durotomy. The present invention provides an effective article of manufacture and related method for sealing an incursion such as a durotomy. The scope of the invention is defined by the appended claims and reasonable equivalents.

What is claimed is:

1. A method for sealing an incursion comprising the steps of:
securing a flange to a first end of a suture;
inserting a second end of the suture into matter surrounding the incursion;
drawing the flange onto the matter in a way that covers the incursion with the flange; and
securing the second end of the suture to the flange,
wherein the flange is formed of a material selected to establish a watertight seal of the incursion when the flange is applied to the matter over the incision.

2. The method as claimed in claim 1 wherein the incursion is a durotomy and the matter is the dura.

3. The method as claimed in claim 1 further comprising the step of inserting one or more supplemental components between the flange and the matter prior to drawing the flange onto the matter.

4. The method as claimed in claim 3 wherein the one or more supplemental components is selected from gel foam, sealant, dural glue and graft.

5. The method as claimed in claim 1 wherein the second end of the suture is secured to the flange with a serrated or toothed component positioned on the suture.

6. The method as claimed in claim 1 wherein the second end of the suture is secured to the flange with a crimp ring positioned on the suture.

7. The method as claimed in claim 1 wherein the second end of the suture is secured to the flange with a bushing.

8. The method as claimed in claim 7 wherein the first end of the suture is secured to the flange with a second bushing.

9. The method as claimed in claim 1 wherein the flange is formed of a nonabsorbent material.

10. The method as claimed in claim 1 wherein the flange is formed of an absorbent material.

11. The method as claimed in claim 1 wherein the flange is formed of a nonmetallic material.

12. The method as claimed in claim 1 wherein the flange is of a selectable shape and size.

13. A method for sealing an incursion comprising the steps of:
securing a securing mechanism to an underlying substrate; and
attaching incursion repair material to the securing mechanism,
wherein the incursion material may be a flange, graft or a combination of the two, and the underlying substrate may be scar tissue or a bone.

14. The method as claimed in claim 13 wherein the securing mechanism is an insertion member arranged to pierce the underlying substrate and the incursion repair material is attached to the insertion member with a suture.

15. The method as claimed in claim 13 wherein the securing mechanism is a screw that is screwed into the underlying substrate, the screw including an eyelet used to attach the incursion repair material to the screw with a suture.

16. The method as claimed in claim 13 wherein the securing mechanism is a clip that is hingedly clipped about the underlying substrate, the clip including an eyelet used to attach the incursion repair material to the clip with a suture.

17. The method as claimed in claim 13 wherein the securing mechanism is a spring-loaded clamp that is clamped about the underlying substrate, the clamp including an eyelet used to attach the incursion repair material to the clamp with a suture.

18. The method as claimed in claim 13 wherein the securing mechanism is a stick inserted into the underlying substrate, wherein the stick establishes a base upon which the incursion material is applied until filling in the incursion.

19. The method as claimed in claim 13 wherein the securing mechanism is a board inserted into the underlying substrate, wherein the board establishes a base upon which the incursion material is applied until filling in the incursion.
